(19) United States
(12) Patent Application Publication
(10) Pub. No.: US 2005/0064362 A1
(43) Pub. Date: Mar. 24, 2005

(54) Dental inspection mirror

(76) Inventors: Marcia Loft, Spokane, WA (US); Ardean Nickerson, Spokane, WA (US)

Correspondence Address: John S. Reid;Reidlaw, L.L.C., 1926 S. Valleyview Lane, Spokane, WA, 99212-0157, US

(21) Appl. No.: 10/944,490

(22) Filed: Sep. 17, 2004

Related U.S. Application Data
(60) Provisional application No. 60/504,632, filed on Sep. 18, 2003.

(51) Int. Cl. A61B 1/24

(52) U.S. Cl. 433/30

(57) ABSTRACT

A dental inspection apparatus includes a first rectangular elongate body portion and a second rectangular elongate body portion. The first body portion is defined by a first top surface, a first bottom surface a first body length, a first body width and a first body thickness. The second body portion is defined by second top surface, a second bottom surface, a second body length, a second body width and a second body thickness. The first body portion is joined to the second body portion approximately at a midpoint of the length of the second body portion such that the first body portion is substantially perpendicular to the second body portion and such that the first top surface and the second top surface form a contiguous joined top surface. The joined first and second body portions define one or more contiguous reflective surfaces substantially reflective to impinging light.

### CROSS REFERENCE TO RELATED APPLICATIONS

This application claims priority to U.S. Provisional Patent Application Ser. No. 60/504,632, filed Sep. 18, 2003 and entitled, “Dental Inspection Mirror”, which is hereby incorporated herein by reference in its entirety.

### BACKGROUND

This invention relates to improvements in the field of dentistry. In particular, this invention relates to an improved dental mirror.

In dentistry, there are relatively few choices when it comes to mouth mirrors. Essentially, such mirrors are limited to those having a round mirrored surface. This configuration has a number of advantages. It is easy and inexpensive to make. It also allows the mirror to be used in a wide range of dental applications. This configuration also has a number of drawbacks.

One drawback with conventional dental mouth mirrors is that the number of teeth which may be viewed using a conventional round mirror is extremely limited. For example, use of a conventional round mirror to view the abutment teeth during bridge preparation allows viewing of only 2 to 2½ teeth. This limited field of vision requires the operator to move the mirror axially with respect to the handle, in order to view the necessary surfaces of the teeth required for bridge preparation. Of course, the operator cannot see all the teeth involved in bridge preparation at once.

Another drawback with conventional round dental mouth mirrors is that there is no way that an operator can determine, through the use of such mirror alone, whether the process of reduction of teeth has reached the desired level, such as when preparing abutment teeth for a level. For example, in preparing the abutment teeth for a bridge, the anterior and posterior abutments of the abutment teeth must be reduced to parallelism in order for the bridge to fit. Using a conventional mouth mirror, the reduction process must be halted, the mouth mirror must be removed from the mouth of the patient and the parallelism must be determined by placing the bridge or a template into the patient's mouth and into the desired location. If the abutment surfaces are not parallel, more reduction is needed and the process must be repeated for successive iterations until parallelism is reached. This trial and error process is time consuming and can be extremely fatiguing for the operator and the patient.

Another example of the drawbacks of conventional dental mouth mirrors is in cosmetic dentistry, such as the application of porcelain anterior veneer facings. In that particular process, the operator must reduce the labial surfaces of the anterior teeth in a uniform manner to accept a uniform application of laminants. Use of a conventional round mouth mirror does not enable the operator to see all six of the anterior teeth. In addition, the operator has no guide by which to reduce the labial surfaces in a uniform manner. Accordingly, the operator must reduce a particular tooth surface by guessing. Then the operator must move the mirror so as to view various portions of the anterior teeth for comparison. Only by performing a number of iterations of this process can the operator achieve uniform reduction.

Furthermore, in training and instructing dental care professionals, it is often desirable that a number of students be able to observe the teeth of a tutorial subject at the same time. As will be appreciated, the conventional round mouth mirror allows only limited viewing by a limited number of students. Another disadvantage of the conventional round mirror includes several ergonomic flaws that can cause back, neck and wrist strain due to the pinch force required to hold and manipulate the mirror in a care-giving or teaching situation. Another disadvantage includes the small surface area of the conventional round mirror capable of reflecting light directed at the area being worked on.

Therefore, the conventional round mouth mirror of the prior art suffers from three identifiable disadvantages. First, its field of view is limited. Second, it has no reference means by which the operator can determine when and if tooth reduction has achieved the desired state. Third, it requires the operator to move the mirror handle axially to change the field of view. It would be desirable, therefore, to provide a dental appliance that overcomes these and other disadvantages.

### SUMMARY

According to an embodiment of the present invention, a dental inspection mirror comprises a first rectangular elongate body portion and a second rectangular elongate body portion. The first body portion is defined by a first top surface, a first bottom surface a first body length, a first body width and a first body thickness. The second body portion is defined by second top surface, a second bottom surface, a second body length, a second body width and a second body thickness. The first body portion is joined to the second body portion approximately at a midpoint of the length of the second body portion such that the first body portion is substantially perpendicular to the second body portion and such that the first top surface and the second top surface form a contiguous joined top surface. The joined first and second body portions define one or more contiguous reflective surfaces substantially reflective to impinging light.

According to another embodiment, a mirror apparatus comprises a mirror body base section, and a mirror body head section. The mirror body base section is defined by a substantially rectangular elongate vertical portion comprising material of a thickness to be substantially rigid and forming opposite facing base section surfaces. The mirror body head section is defined by a substantially rectangular elongate horizontal portion comprising material of a thickness to be substantially rigid and comprising opposite facing head section surfaces and that is perpendicular to the mirror body base section. The mirror body base section is joined to the second body portion approximately at the midpoint of the first elongate portion. The mirror apparatus is substantially reflective on at least one of the opposite facing surfaces.

According to still another embodiment, a dental appliance comprises first body portion means and body portion means. The first body portion means is defined by a first top surface, a first bottom surface, a first body length, a first body width and a first body thickness. The second body portion means is defined by second top surface, a second bottom surface, a second body length, a second body width and a second body thickness. The first body portion means is joined to the second body portion means approximately at a midpoint of the length of the second body portion means such that the first body portion means is substantially perpendicular to the second body portion means and such that the first top surface and the second top surface form a contiguous joined top surface. A contiguous surface of the joined first and second and second body portion means is substantially reflective to impinging light.

The foregoing, and other features and advantages of the invention, will become further apparent from the following detailed description of the presently disclosed embodiments read in conjunction with the accompanying drawings.

### DESCRIPTION OF THE DRAWINGS

FIG. 1 is a plan view depicting a dental inspection mirror, illustrating an embodiment of the present invention.

FIG. 2 is a perspective view of the dental inspection mirror of FIG. 1.

FIG. 3 is a plan view depicting a mirror apparatus, illustrating another embodiment of the present invention.

FIG. 4 is a plan view depicting a dental inspection mirror that is pivotally joined, illustrating yet another embodiment of the present invention.

FIG. 5 is an exploded perspective view of the dental inspection mirror of FIG. 4.

### DETAILED DESCRIPTION

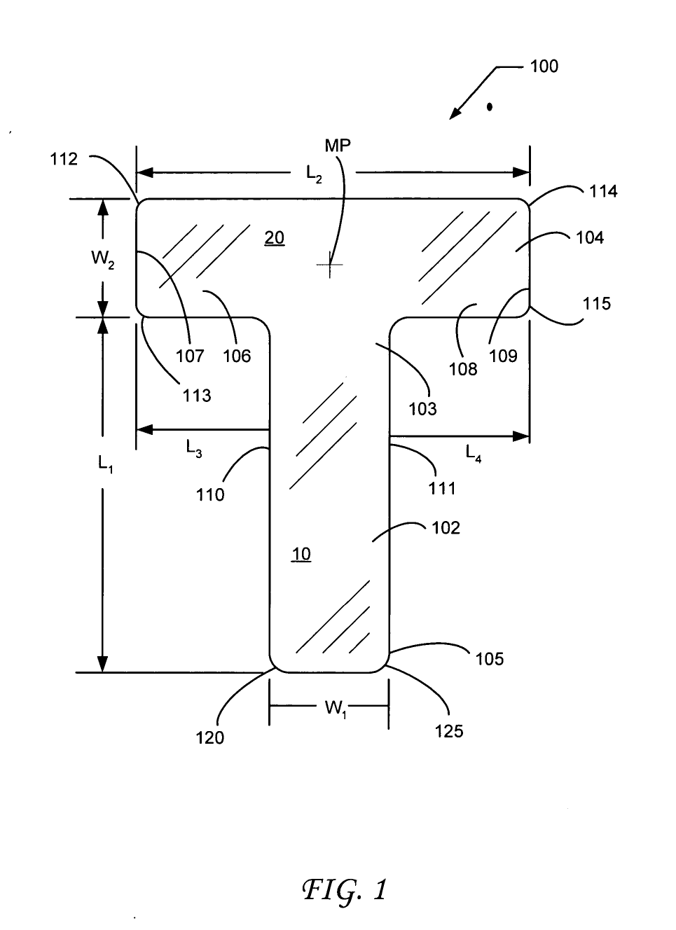

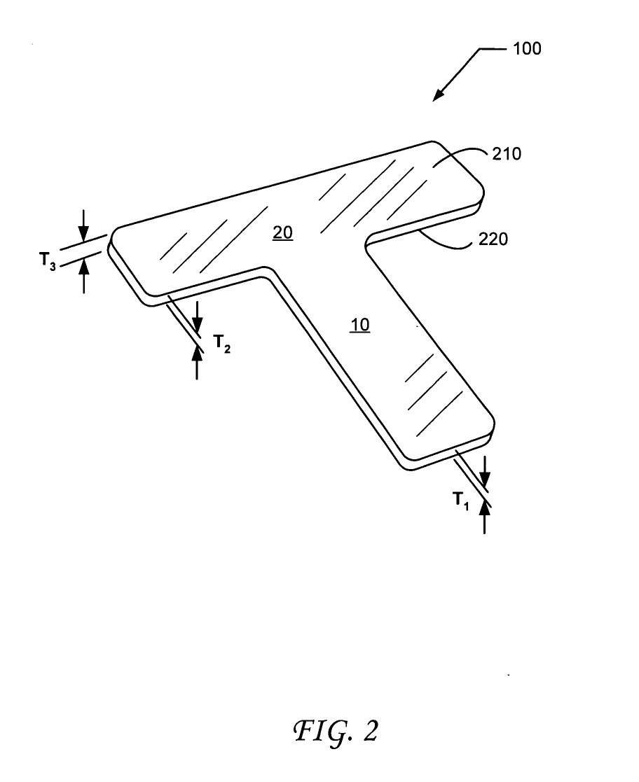

FIG. 1 is a plan view diagram of a dental inspection mirror, illustrating an embodiment of the present invention. FIG. 1 depicts a generally “T” shaped dental inspection mirror 100. Dental inspection mirror 100 includes a first rectangular elongate body portion 102 and a second rectangular elongate body portion 104. The first body portion 102 (mirror body base section) is defined by a first top surface 10, a first bottom surface (not visible), a first body length (L₁), a first body width (W₂) and a first body thickness (T₁), (as shown in FIG. 2). The second body portion 104 (mirror body head section) is defined by a second top surface 20, a second bottom surface (not visible), a second body length (L₁), a second body width (W₂), and a second body thickness (T₂) (as is shown in FIG. 2). As illustrated in FIG. 1, the first body portion 102 is joined to the second body portion 104 approximately at a midpoint (MP) of the length of the second body portion 104 such that the first body portion 102 is substantially perpendicular to the second body 104 portion and such that the first top surface 10 and the second top surface 20 form a contiguous joined top surface. Furthermore, as illustrated in FIG. 1, the joined first and second body portions (102, 104) define one or more contiguous reflective surfaces substantially reflective to impinging light. In one embodiment, as illustrated in FIG. 1, the second body portion 104 thus defines “wings” or “ears” 106, 108 either side of the main body portion 102. As can also be seen in FIG. 1, the corners of dental inspection mirror 100 are generally rounded to aid in comfort of the user and patients. The first and second body lengths (L₁, L₂) can be any suitable length appropriate for a dental instrument. The first and second body widths (W₁, W₂) can be any dimension suitable for dental inspection. In one embodiment, the first and second body widths (W₁, W₂) are between about 10% and 50% of the first body length (L₁).

In one embodiment, the first body portion 102 is defined by a junction end 103 where the first body portion 102 is joined to the second body portion 104, and a base end 105 opposite the junction end along the second body portion 104 length (L₂). In another embodiment, the first body portion 102 is further defined by corners (120, 125) at the base end, and one or more of the corners at the base end are radiused. In another embodiment, the second body portion 104 is defined by a left side extension 106 and a right side extension 108, each extension defined by an extension length (L₃, L₄) that extends from left and right outer edges (110, 111) of the first body portion 102 to left and right outer edges (107, 109) of the second body portion 106. The left and right extension lengths (L₃, L₄) can be any length suitable for dental inspection. In one embodiment, the left and right extension lengths (L₃, L₄) are between about 25% and 75% of the first body length (L₁).

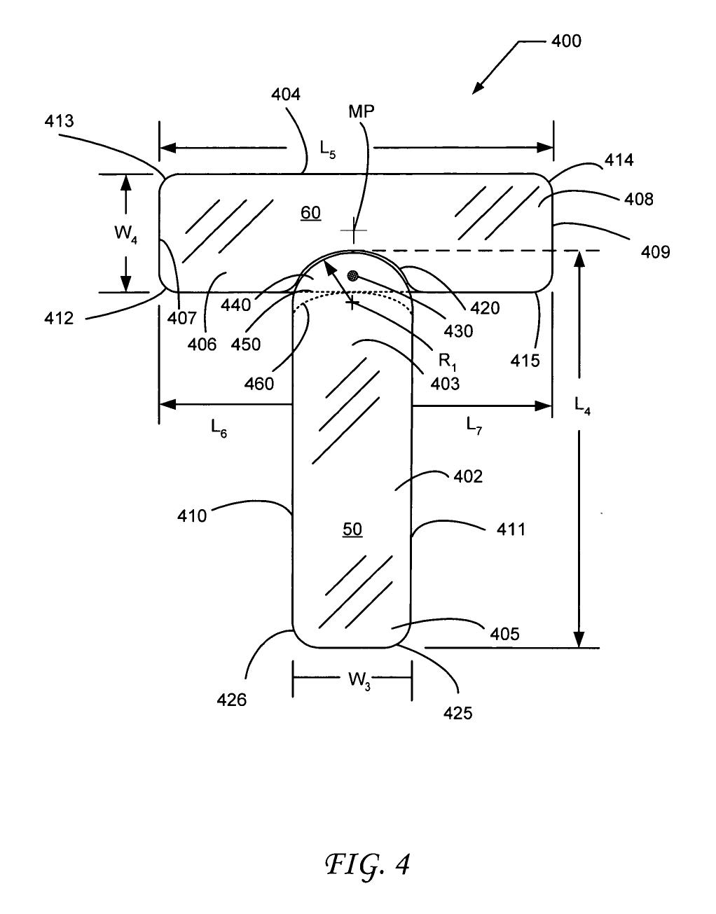

In yet another embodiment, the right side extension 108 and the left side extension 106 are defined by left outer corners (112, 113) and right outer corners (114, 115), and one or more of the outer corners are radiused. In one embodiment, as shown in FIG. 1, the mirror 100 is formed as a unitary piece. In another embodiment, (e.g., as is shown in FIG. 4) the first body portion 102 is pivotally joined to the second body portion 104. In another embodiment, the first and second body portions (102, 104) are composed of a material such as tempered glass, metal, plastic and polymer. In one such embodiment, the plastic, polymer and glass are coated on the top and/or bottom surfaces with a substantially reflective material. In another such embodiment, the metal material is polished on at least a one of the top and bottom surface to a substantially reflective finish. In yet another embodiment, the thickness of the first and second body portions (102,104) is between about 0.0675 and 0.3125 inches. In another embodiment, the thickness of the dental mirror 100 varies in one or more directions to provide additional enhancements, such as, for example optical surface curvature, and improved flexibility.

FIG. 2 is a perspective view of the dental inspection mirror of FIG. 1. As shown in FIG. 2, dental inspection mirror 100 includes a unitary top surface 210, and a unitary bottom surface 220 (not visible in FIG. 2). In one embodiment, the unitary top surface 210, and the unitary bottom surface 220 are formed by the union of first and second body portions (102, 104, in FIG. 1) that have first and second top surfaces (10, 20). As illustrated in FIG. 2, first and second top surfaces (10, 20) form at least one contiguous reflective surface (e.g., 210, 220) that is substantially reflective to impinging light. FIG. 2 further shows the dental inspection mirror 100 having a first thickness T. (corresponding to the first body portion 102 of FIG. 1) a second thickness T₂ (corresponding to the second body portion 104 of FIG. 1) and a third thickness T₃ that is different from the second thickness (T₂) to illustrate an embodiment having variable thickness in the second body portion 104 of FIG. 1. In one embodiment, the thickness (e.g., T₁, T₂) of the dental inspection mirror 100 is between about 0.0675 and 0.3125 inches. In another embodiment, the thickness of the dental inspection mirror 100 varies in one or more directions (e.g., axially or longitudinally) to provide additional functionality, such as, for example optical surface curvature, and improved flexibility or stiffness. For example, FIG. 2 illustrates a variable thickness along the width of the second body portion 104 of FIG. 1 as the second body portion body transitions from the first thickness (T₂) to the second thickness (T₃). The thickness of dental inspection mirror 100 can be varied in any manner that provides beneficial properties.

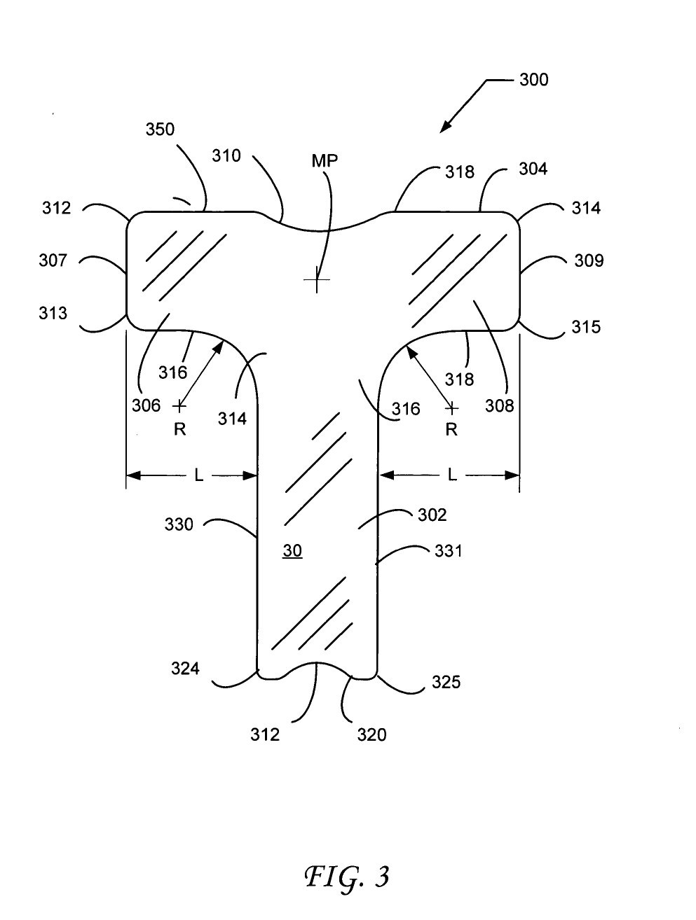

FIG. 3 is a plan view depicting a mirror apparatus, illustrating yet another embodiment of the present invention. FIG. 3 depicts a generally “T” shaped mirror apparatus 300 similar to the dental inspection mirror of FIGS. 1 and 2.

As with dental inspection mirror 100, mirror apparatus 300 includes a first rectangular elongate body portion 302 and a second rectangular elongate body portion 304, each defined by top surface and bottom surfaces, body lengths, body widths and body thicknesses as described with reference to FIGS. 1 and 2. As illustrated in FIG. 3, the first body portion 302 is joined to the second body portion 304 approximately at a midpoint “MP” of the length of the second body portion 304 such that the first body portion 302 is substantially perpendicular to the second body 304 portion and such that the first top surface and the second top surface form a contiguous joined top surface (30). Furthermore, as illustrated in FIG. 3, the joined first and second body portions (302, 304) define one or more contiguous reflective surfaces substantially reflective to impinging light.

In another embodiment, the first body portion 302 is further defined by corners (324, 325) at the base end 320, and one or more of the corners at the base end 320 are radiused. In yet another embodiment, the second body portion 304 is defined by a left side extension 306 and a right side extension 308, each extension defined by an extension length “L” that extends from left and right outer edges (330,331) of the first body portion 302 to left and right outer edges (316, 318) of the second body portion 304. In still another embodiment, the right side extension 308 and the left side 306 extension are defined by left outer corners (312, 313) and right outer corners (314, 315), and one or more of the outer corners are radiused. In one embodiment, the radius “R” in the left and right arc junction regions (314, 316) is between about 50% and 100% of the length “L” of the left and right extension (306, 308). In one embodiment, (not shown) the first body portion 302 is pivotally joined to the second body portion 304. In still another embodiment, the second body portion 304 is further defined by a reverse radius 310 centered along a top edge (350) of the second body portion and extending for a portion of the top edge (350) between outer corners (312, 314) of the left and right extensions (306, 308).

In one embodiment, the first body portion 302 is defined by a junction end (not numbered) where the first body portion 302 is joined to the second body portion 304 and a base end 320 opposite the junction end along the length of the second body portion 304. In another embodiment, the first body portion 302 is further defined by opposite corners (324, 325) at the base end 320, and the base end is defined by a reverse radius 312 extending between the opposite corners (324, 325) of the base end. In yet another embodiment, the junction end defines a radiused junction region such that the first body portion 302 is joined to the second body portion 304 along a left side arc junction region 314 and a right side arc junction region 316. The junction regions (314, 316) extend along left and right radii (R) symmetrically from left and right outside edges of the first body portion (330, 331) to the left and right second body portion extensions (306, 308).

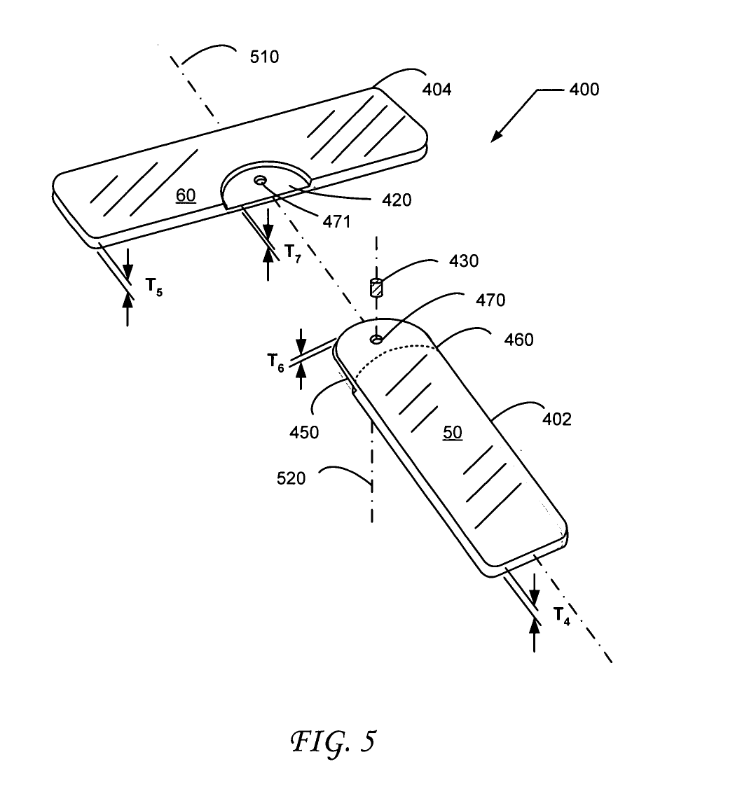

FIG. 4 is a plan view depicting a dental inspection mirror that is pivotally joined, illustrating yet another embodiment of the present invention. FIG. 4 again depicts a generally “T” shaped dental inspection mirror 400. Dental inspection mirror 400 again includes a first rectangular elongate body portion 402 and a second rectangular elongate body portion 404. The first body portion 402 is again defined by a first top surface 50, a first bottom surface (not visible), a first body length (L₄), a first body width (W₃) and two first body thicknesses (T₃,T₅) (as shown in FIG. 5). The second body portion 404 is defined by a second top surface 60, a second bottom surface (not visible), a second body length (L₁), a second body width (W₂), and two second body thicknesses (T₄,T₆) (as shown in FIG. 5).

As illustrated in FIG. 4, the first body portion 402 is pivotally joined to the second body portion 404 approximately at a midpoint (MP) of the length of the second body portion 404 such that the first body portion 402 is substantially perpendicular to the second body 104 portion and such that the first top surface 50 and the second top surface 60 form a generally contiguous pivotally joined top surface. Furthermore, as illustrated in FIG. 4, the pivotally joined first and second body portions (402, 404) define one or more contiguous reflective surfaces substantially reflective to impinging light. As in the embodiments of FIGS. 1-3, the second body portion 104 of dental inspection mirror 400 defines “wings” or “ears” (406, 408) on either side of the main body portion 402. As can also be seen in FIG. 1, the corners of dental inspection mirror 400 are again generally rounded to aid in comfort of the user and patients. The first and second body lengths (L₄, L₅) can be any suitable length appropriate for a dental instrument. The first and second body widths (W₃, W₄) can be any dimension suitable for dental inspection. In one embodiment, the first and second body widths (W₃, W₄) are between about 10% and 50% of the first body length (L₄).

In one embodiment, the first body portion 402 is defined by a junction end 403 where the first body portion 402 is pivotally joined to the second body portion 404 with a pin 430 or a suitable equivalent. The junction end 403 is further defined by a generally semi-circular top 440 having a radius (R₁) that rotates around the pin 430 when the first body portion is positioned within a generally semi-circular shelved region 420 formed in the second body portion 404 to receive the junction end 403. In another embodiment, the first body region is further defined by a shelved region 450 having less thickness in a region that follows an arc 460 (visible as a hidden line) on the first bottom surface of the first body portion 402. In another embodiment, the first body portion 402 is defined by a base end 405 opposite the junction end 403 along the second body portion 404 length (L₄). In another embodiment, the first body portion 102 is further defined by corners (425, 426) at the base end 405, and one or more of the corners (425, 426) at the base end are radiused. In another embodiment, the second body portion 404 is defined by a left side extension 406 and a right side extension 408, each extension defined by extension lengths (L₆, L₇) that extend from left and right outer edges (410, 411) of the first body portion 402 to left and right (407, 409) outer edges of the second body portion 406. The left and right extension lengths (L₆, L₇) can be any length suitable for dental inspection. In one embodiment, the left and right extension lengths (L₆, L₇) are between about 25% and 75% of the first body length (L₄). In yet another embodiment, the second body portion is further defined by a semi-circular shelved region 420 having less thickness in a region that follows a radius (R₁) on the top surface of the second body portion 404. In one embodiment, (visible in FIG. 5) the shelved regions (450, 420) of the first and second body portions include through-holes for the cotter pin 430. In one embodiment, the radius (R₁) is about 50% of the first body portion width (W₃).

In yet another embodiment, the right side extension 408 and the left side extension 406 are defined by left outer corners (412, 413) and right outer corners (414, 415), and one or more of the outer corners are radiused. In another embodiment, the first and second body portions (402, 404) are composed of a material such as tempered glass, metal, plastic and polymer. In one such embodiment, the plastic, polymer and glass are coated on the top and/or bottom surfaces with a substantially reflective material. In another such embodiment, the metal material is polished on at least a one of the top and bottom surface to a substantially reflective finish. In yet another embodiment, the thickness of the first and second body portions (402,404) is between about 0.0675 and 0.3125 inches. In another embodiment, the thickness of the dental mirror 400 varies in one or more directions to provide additional enhancements, such as, for example optical surface curvature, and improved flexibility.

FIG. 5 is an exploded perspective view of the dental inspection mirror of FIG. 4. As shown in FIG. 5, dental inspection mirror 400 includes first and second top surfaces (50, 60). In one embodiment, the first top surface 50, and the second top surface 60 are united by pivotally joining first and second body portions (402, 404, in FIG. 4) along a first axis 510 and a second axis 520. As illustrated in FIG. 5, first and second top surfaces (50, 60) form at least one substantially contiguous reflective surface that is substantially reflective to impinging light when the first and second body portions are joined.

FIG. 5 further shows the dental inspection mirror 400 having a first thickness T₄ corresponding to the first body portion 402 of FIG. 4. A second thickness T₅, is also shown that corresponds to the second body portion 404 of FIG. 4. A third thickness (T₆) is also depicted in FIG. 5 corresponding to a generally semi-circular shelved region 450 having an outer radius (approximately R₁, as shown in FIG. 4) and a lower arc boundary 460. A fourth thickness (T₇) that defines a semi-circular shelved region 420 having less thickness in a region that follows the radius (R₁) on the top surface of the second body portion 404 is shown. FIG. 5 also shows a pin 430 that is on a centerline 520 of a first body 402 through-hole 470 within the generally semi-circular shelved region 450 between the outer radius (approximately R₁, as shown in FIG. 4) and the lower arc boundary 460 (visible as a hidden line). A second through-hole 471 is shown on the second body portion 404 within the semi-circular shelved region 420. As shown in FIG. 5, pin 430 is fitted in through-hole 470 of the first body portion 402 and also in through-hole 470 of the second body portion 404 to allow rotation of the second body portion 404 about the axis of 520 relative to the first body portion. However, it should be understood that the first body portion 402 and the second body portion can be pivotally joined by any suitable mechanical coupling that provides pivoting of the first body portion 402 to the second body portion 404.

In one embodiment, the thickness (e.g., T₄, T₅) of one portion of the dental inspection mirror 400 is between about 0.0675 and 0.3125 inches. In another embodiment, the thickness (e.g., T₆, T₆) of another portion of the dental inspection mirror 400 is between about 0.03375 and 0.15625 inches. As with the embodiments of FIGS. 1-3, the thickness of dental inspection mirror 400 can be varied in whole or in part in any manner that provides beneficial properties.

In some embodiments, the first and second body portions (402, 404) are composed of a material such as tempered glass, metal, plastic and polymer. In one such embodiment, the plastic, polymer and glass are coated on the top and/or bottom surfaces with a substantially reflective material. In another such embodiment, the metal material is polished on at least a one of the top and bottom surface to a substantially reflective finish. In yet another such embodiment, the thickness of the first and second body portions (402, 404) is between about 0.0675 and 0.3125 inches. In still another such embodiment (not shown), the thickness of the dental mirror 400 varies in one or more directions to provide additional enhancements, such as, for example optical surface curvature, and improved flexibility.

Another embodiment (not shown) comprises a mirror apparatus that includes a mirror body base section, and a mirror body head section. In the present embodiment, the mirror body base section is defined by a substantially rectangular elongate vertical portion comprising material of a thickness to be substantially rigid and forming opposite facing base section surfaces. In another embodiment, the mirror body head section is defined by a substantially rectangular elongate horizontal portion comprising material of a thickness to be substantially rigid and comprising opposite facing head section surfaces and that is perpendicular to the mirror body base section. In yet another embodiment, the mirror body base section is joined to the second body portion approximately at the midpoint of the first elongate portion. In still another embodiment, the mirror apparatus is substantially reflective to impinging light on at least one of the opposite facing surfaces. In one variation, the mirror body base section is joined to the second body portion at a location distal from the midpoint of the first elongate portion, to thereby form a more “L”-shaped mirror, or a mirror having a geometry approximately between a “T” shape and an “L” shape.

In some embodiments, materials of construction of mirrors of FIGS. 1-3 include, without limitation: clear transparent plastic, (e.g., thermoplastic) having a reflective coating on one side (preferably applied to the back side of the plastic with the reflective material thus being presented directly to the plastic); mirrored glass; polished metal; and other highly reflective, relatively stiff materials. Preferably the mirror is between about 1/16 of an inch and about 5/16 of an inch in thickness (cross sectional thickness), although the final thickness can vary based upon the material of construction. Generally, the mirror is sufficiently thick (in cross section) to provide stiffness to the mirror so that it does not easily deflect when in use, but is not so thick that it interferes with the dental inspection process of results in patient discomfort.

Among other advantages, one or more of the above described embodiments provide improved ergonomics for dental professionals (operators, instructors and students) including a reduction in the required pinch force to hold and utilize the mirror, as well as reduced back, neck and wrist strain. Further, the increased illumination of the working field due to the greater surface of mirror exposed to reflecting light directed at the area which is particularly helpful for detail work and photography. Yet another advantage of one or more of the above described embodiments includes mirror geometries and surface area that more effectively retract the cheeks, lips and tongue. For example, when teaching radiography, one or more of the above embodiments can be used to view the occlusion of the maxillary and mandibular arches to achieve proper alignment of the film placement.

While the apparatus disclosed and claimed herein is described as being particularly useful to dental inspection applications, it will be appreciated that the apparatus can be used in additional applications, such as automotive applications, etc.

It is understood that the invention can be embodied in other specific forms not described that do not depart from its spirit or essential characteristics. The described embodiments are to be considered in all respects only as illustrative and not restrictive, the scope of the invention being defined by the appended claims and equivalents thereof.

1. A dental inspection mirror, comprising:
a first rectangular elongate body portion and a second rectangular elongate body portion, and wherein:
the first body portion is defined by a first top surface, a first bottom surface a first body length, a first body width and at least a first body thickness;
the second body portion is defined by second top surface, a second bottom surface, a second body length, a second body width and at least a second body thickness;
the first body portion is joined to the second body portion approximately at a midpoint of the length of the second body portion such that the first body portion is substantially perpendicular to the second body portion and such that the first top surface and the second top surface form a contiguous joined top surface; and
the joined first and second body portions define one or more contiguous reflective surfaces substantially reflective to impinging light.

2. The dental inspection mirror of claim 1, and wherein the first body portion is defined by a junction end where the first body portion is joined to the second body portion and a base end opposite the junction end along the second body portion length.

3. The dental inspection mirror of claim 2, and wherein the first body portion is further defined by corners at the base end, and one or more of the corners is radiused.

4. The dental inspection mirror of claim 2, and wherein the first body portion is further defined by opposite corners at the base, and the base end is defined by a reverse radius extending between the opposite corners.

5. The dental inspection mirror of claim 2, and wherein the junction end defines a radiused junction region such that the first body portion is joined to the second body portion along a left side arc junction region and a right side arc junction region extending generally symmetrically in opposing direction from the first to the second body portions along left and right radii perpendicular to the first body portion and the second body portion.

6. The dental inspection mirror of claim 1, and wherein the second body portion is defined by a left side extension and a right side extension, each extension defined by an extension length that extends from an outer edge of the first body portion to left and right outer edges of the second body portion.

7. The dental inspection mirror of claim 5, and wherein the radius is between about 50% and 100% of the length of the right and left side extensions.

8. The dental inspection mirror of claim 6, and wherein the right side extension and the left side extension are defined by outer corners, and further wherein one or more of the outer corners are radiused.

9. The dental inspection mirror of claim 6, and wherein the second body portion is defined by a reverse radius extending between the outer edges of the left and right extensions and that is opposite of the first body portion.

10. The dental inspection mirror of claim 1, and wherein the first body portion is pivotally joined to the second body portion.

11. The dental inspection mirror of claim 1, and wherein the first and second body portions are composed of a material selected from the group consisting of: tempered glass, a metal, a plastic and a polymer.

12. The dental inspection mirror of claim 11, and wherein the plastic and polymer are coated on at least one of the top and bottom surfaces with a reflective material.

13. The dental inspection mirror of claim 11, and wherein the glass material is coated on at least one of the top and bottom surfaces with a reflective material.

14. The dental inspection mirror of claim 11, and wherein the metal material polished on at least a one of the top and bottom surface to a substantially reflective finish.

15. The dental inspection mirror of claim 1, and wherein the thickness of the first and second body portion is between about 0.0675 and 0.3125 inches.

16. The dental inspection mirror of claim 15, and wherein the thickness is variable along a dimension selected from the group consisting of: the first body length, the first body width, the second body length and the second body width.

17. A mirror apparatus, comprising:
a mirror body base section, and a mirror body head section, and wherein:
the mirror body base section is defined by a substantially rectangular elongate vertical portion comprising material of a thickness to be substantially rigid and forming opposite facing base section surfaces;
the mirror body head section is defined by a substantially rectangular elongate horizontal portion comprising material of a thickness to be substantially rigid and comprising opposite facing head section surfaces and that is perpendicular to the mirror body base section;
the mirror body base section is joined to the second body portion approximately at the midpoint of the first elongate portion; and
the mirror apparatus is substantially reflective on at least one of the opposite facing surfaces.

18. The mirror apparatus of claim 17, and wherein the mirror body base section and the mirror body head section material are selected from the group consisting of: tempered glass, a metal, a plastic and a polymer.

19. The mirror apparatus of claim 17, and wherein the thickness of the mirror body base section and mirror body head section is between about 0.0675 and 0.3125 inches.

20. A dental appliance, comprising:
first body portion means and second body portion means, and wherein:
the first body portion means is defined by a first top surface, a first bottom surface, a first body length, a first body width and a first body thickness;
the second body portion means is defined by second top surface, a second bottom surface, a second body length, a second body width and a second body thickness;
the first body portion means is joined to the second body portion means approximately at a midpoint of the length of the second body portion means such that the first body portion means is substantially perpendicular to the second body portion means and such that the first top surface and the second top surface form a contiguous joined top surface; and
a contiguous surface of the joined first and second and second body portion means is substantially reflective to impinging light.
